(19) United States
(12) Patent Application Publication
(10) Pub. No.: US 2003/0092739 A1
(43) Pub. Date: May 15, 2003

(54) Heterocyclic compounds

(76) Inventors: Chun-Lin Yeh, Taichung (TW); Chien-Hsing Chen, Taichung (TW)

Correspondence Address: BACON & THOMAS, PLLC, 625 SLATERS LANE, FOURTH FLOOR, ALEXANDRIA, VA, 22314

(21) Appl. No.: 09/886,158

(22) Filed: Jun. 22, 2001

(51) Int. Cl. A61K 31/4439; A61K 31/44; A61K 31/427; C07D 417/02; C 07D 4 1/02

(52) U.S. Cl. 514/338; 514/277; 514/365; 546/273.4; 546/339; 548/181

(57) ABSTRACT

The present invention relates to a novel heterocyclic compound having the formula:

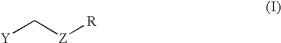

wherein Y represents a 5- or 6-membered nitrogen-containing heterocyclic ring; Z represents a nitrogen atom, an oxygen atom, a sulfur atom or NR¹; and R and R¹ are as defined in the description. These heterocyclic compounds are useful as pesticides.

### FIELD OF THE INVENTION

The present invention is related to heterocyclic compounds useful as pesticides.

### BACKGROUND OF THE INVENTION

Certain heterocyclic compounds have been described as useful as pesticides. For example, U.S. Pat. No. 4,742,060 discloses a heterocyclic compound of the formula:

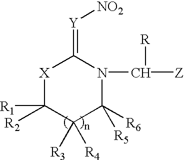

wherein n=0 or 1; X=S, O, —N—R₇ or —CH—R₈; Y∇N or —C—R₉; R₇, R₈, R₉=hydrogen or specific organic radicals; Z=5- or 6-membered nitrogen-containing heterocyclic ring ; R₁, R₂, R₅, R₆=hydrogen or alkyl group ; R₃, R₄=hydrogen, hydroxy group or alkyl group. U.S. Pat. No. 4,849,432 discloses a heterocyclic compound similar to the foregoing heterocyclic compound, except that the group —NO₂ is replaced by a —CN group and that Y is a nitrogen group. The above heterocyclic compounds are useful as pesticides.

It is a purpose of the present invention to provide novel heterocyclic compounds useful as pesticides.

### SUMMARY OF THE INVENTION

The present invention relates to a novel heterocyclic compound having the formula:

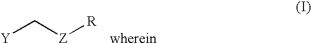

Y represents a 5- or 6-membered nitrogen-containing heterocyclic ring;

Z represents a nitrogen atom, an oxygen atom, a sulfur atom or NR¹;

R represents a hydrogen atom, a halogen atom, a hydroxy group, an alkoxy group, a substituted or unsubstituted amino group, a cyano group, a nitro group, a nitroso group, an alkyl group, an alkenyl group, an alkynyl group, a substituted or unsubstituted phenyl or naphthyl group, an alkylcarbonyl group, an alkenylcarbonyl group, an alkynylcarbonyl group, a substituted or unsubstituted phenylcarbonyl group, an alkoxycarbonyl group, an alkylthiocarbonyl group, a substituted or unsubstituted phenoxycarbonyl group, a substituted or unsubstituted phenylthiocarbonyl group, a monoalkyl- or dialkyl-aminocarbonyl group, a substituted or unsubstituted phenylaminocarbonyl group, a substituted or unsubstituted benzoylaminocarbonyl group, an alkylsulfonyl group, a substituted or unsubstituted phenylsulfonyl group, a substituted or unsubstituted phenylsulfonylaminocarbonyl group, an organophosphono group, an organothionophosphono group, or

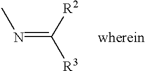

R² and R³ independently represent an alkyl group, an alkenyl group, an alkynyl group, a substituted or unsubstituted phenyl or naphthyl group, an alkoxy group, an alkenyloxy group, an alkynyloxy group, a substituted or unsubstituted phenyloxy or naphthyloxy group, an alkylthio group, an alkenylthio group, an alkynylthio group, a substituted or unsubstituted phenylthio or naphthylthio group, a substituted or unsubstituted amino group.

R¹ represents a hydrogen atom, a halogen atom, a hydroxy group, an alkoxy group, a substituted or unsubstituted amino group, a cyano group, a nitro group, a nitroso group, an alkyl group, an alkenyl group, an alkynyl group, a substituted or unsubstituted phenyl or naphthyl group, an alkylcarbonyl group, an alkenylcarbonyl group, an alkynylcarboyl group, a substituted or unsubstituted phenylcarbonyl group, an alkoxycarbonyl group, an alkylthiocarbonyl group, a substituted or unsubstituted phenoxycarbonyl group, a substituted or unsubstituted phenylthiocarbonyl group, a monoalkyl- or dialkyl-aminocarbonyl group, a substituted or unsubstituted phenylaminocarbonyl group, a substituted or unsubstituted benzoylaminocarbonyl group, an alkylsulfonyl group, a substituted or unsubstituted phenylsulfonyl group, a substituted or unsubstituted phenylsufonylaminocarbonyl group, an organophosphono group, an organothionophosphono group, or

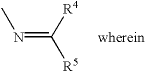

R⁴ and R⁵ independently represent an alkyl group, an alkenyl group, an alkynyl group, a substituted or unsubstituted phenyl or naphthyl group, an alkoxy group, an alkenyloxy group, an alkynyloxy group, a substituted or unsubstituted phenyloxy or naphthyloxy group, an alkylthio group, an alkenylthio group, an alkynylthio group, a substituted or unsubstituted phenylthio or naphthylthio group, a substituted or unsubstituted amino group.

These heterocyclic compounds are useful as pesticides.

### DETAILED DESCRIPTION OF THE INVENTION

The present invention relates to a novel heterocyclic compound having the formula:

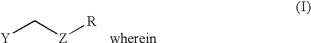

Y represents a 5- or 6-membered nitrogen-containing heterocyclic ring;

Z represents a nitrogen atom, an oxygen atom, a sulfur atom or NR¹;

R represents a hydrogen atom, a halogen atom, a hydroxy group, an alkoxy group, a substituted or unsubstituted amino group, a cyano group, a nitro group, a nitroso group, an alkyl group, an alkenyl group, an alkynyl group, a substituted or unsubstituted phenyl or naphthyl group, an alkylcarbonyl group, an alkenylcarbonyl group, an alkynylcarbonyl group, a substituted or unsubstituted phenylcarbonyl group, an alkoxycarbonyl group, an alkylthiocarbonyl group, a substituted or unsubstituted phenoxycarbonyl group, a substituted or unsubstituted phenylthiocarbonyl group, a monoalkyl- or dialkyl-aminocarbonyl group, a substituted or unsubstituted phenylaminocarbonyl group, a substituted or unsubstituted benzoylaminocarbonyl group, an alkylsulfonyl group, a substituted or unsubstituted phenylsulfonyl group, a substituted or unsubstituted phenylsulfonylaminocarbonyl group, an organophosphono group, an organothionophosphono group, or

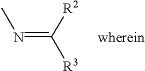

R² and R³ independently represent an alkyl group, an alkenyl group, an alkynyl group, a substituted or unsubstituted phenyl or naphthyl group, an alkoxy group, an alkenyloxy group, an alkynyloxy group, a substituted or unsubstituted phenyloxy or naphthyloxy group, an alkylthio group, an alkenylthio group, an alkynylthio group, a substituted or unsubstituted phenylthio or naphthylthio group, a substituted or unsubstituted amino group.

R¹ represents a hydrogen atom, a halogen atom, a hydroxy group, an alkoxy group, a substituted or unsubstituted amino group, a cyano group, a nitro group, a nitroso group, an alkyl group, an alkenyl group, an alkynyl group, a substituted or unsubstituted phenyl or naphthyl group, an alkylcarbonyl group, an alkenylcarbonyl group, an alkynylcarboyl group, a substituted or unsubstituted phenylcarbonyl group, an alkoxycarbonyl group, an alkylthiocarbonyl group, a substituted or unsubstituted phenoxycarbonyl group, a substituted or unsubstituted phenylthiocarbonyl group, a monoalkyl- or dialkyl-aminocarbonyl group, a substituted or unsubstituted phenylaminocarbonyl group, a substituted or unsubstituted benzoylaminocarbonyl group, an alkylsulfonyl group, a substituted or unsubstituted phenylsulfonyl group, a substituted or unsubstituted phenylsufonylaminocarbonyl group, an organophosphono group, an organothionophosphono group, or

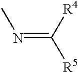

R⁴ and R⁵ independently represent an alkyl group, an alkenyl group, an alkynyl group, a substituted or unsubstituted phenyl or naphthyl group, an alkoxy group, an alkenyloxy group, an alkynyloxy group, a substituted or unsubstituted phenyloxy or naphthyloxy group, an alkylthio group, an alkenylthio group, an alkynylthio group, a substituted or unsubstituted phenylthio or naphthylthio group, a substituted or unsubstituted amino group.

The heterocyclic compounds of formula (I) of the present invention are found to be very effective against insects and can be useful as pesticides.

The following examples illustrate preparation of the heterocyclic compounds of formula (I).

### EXAMPLE 1

### Preparation of 6-chloro-pyridin-3-ylmethyl-cyanamide

A mixture of 50.0 mmol of 6-chloro-3-chloromethyl pyridine (CCMP), 50.0 mmol of cyanamide, 87.5 mmol of potassium carbonate and 100 ml of acetonitrile was refluxed for 1 hour. The salt was filtered out. The most of the acetonitrile was removed from the filtrate by distillation and crude product was obtained. After purification, the pure desired 6-Chloro-pyridin-3-ylmethyl-cyanamide was obtained as light orange solid. The melting point is in a range of from 119 to 120° C.

### EXAMPLE 2

### Preparation of N-(6-chloro-pyridin-3-ylmethoxy)-thioacetimidic Acid Methyl Ester

Prepared as described above in Example 1 except that an equivalent amount of N-hydroxy-thioacetimidic acid methyl ester was used instead of cyanamide.

### EXAMPLE 3

### Preparation of 2-chloro-5-(2-isopropoxy-phenoxymethyl)-pyridine

Prepared as described above in Example 1 except that an equivalent amount of 2-isopropoxy-phenol was used instead of cyanamide.

### EXAMPLE 4

### Preparation of 2-chloro-5-(3,5-dimethyl-4-methylsulfanyl-phenoxymethyl)-pyridine

Prepared as described above in Example 1 except that an equivalent amount of 3,5-dimethyl-4-methylsulfanyl-phenol was used instead of cyanamide.

### EXAMPLE 5

### Preparation of 2-chloro-5-(naphthalen-1-yloxymethyl)-pyridine

Prepared as described above in Example 1 except that an equivalent amount of naphthalen-1-ol was used instead of cyanamide.

### EXAMPLE 6

### Preparation of 2-chloro-5-(2,2-dimethyl-2,3-dihydro-benzofuran-7-yloxymethyl)-pyridine

Prepared as described above in Example 1 except that an equivalent amount of 2,2-dimethyl-2,3-dihydro-benzofuran-7-ol was used instead of cyanamide.

### EXAMPLE 7

### Preparation of 1-[1-(6-chloro-pyridin-3-ylmethyl)-1H-benzoimidazol-2-yl]-3-mehtyl-urea

Prepared as described above in Example 1 except that an equivalent amount of carbenzadim was used instead of cyanamide.

### EXAMPLE 8

### Preparation of 1-[1-(2-chloro-thiazol-4-ylmethyl)-1H-benzoimidazol-2-yl]-3-methyl-urea

Prepared as described above in Example 1 except that an equivalent amount of 2-chloro-4-chloromethyl-thiazole (CCMT) was used instead of 6-chloro-3-chloromethyl pyridine (CCMP) and an equivalent amount of carbenzadim was used instead of cyanamide.

Table 1 lists the formula and the melting point of the final product of each of the foregoing examples.

TABLE 1

| | | Melting |
|---|---|---|
| Example | Product | point, ° C. |
| 1 | 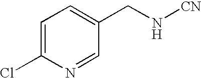 | 119-120 |
| 2 | 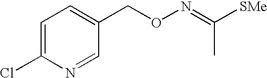 | 68-69 |
| 3 | 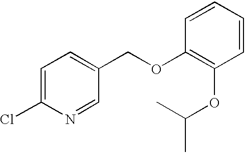 | — |
| 4 | 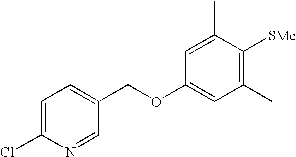 | 59-60 |
| 5 | 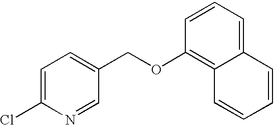 | 84-85 |
| 6 | 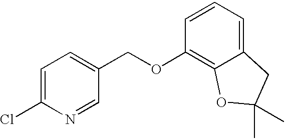 | 56-57 |
| 7 | 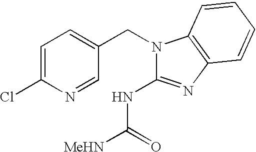 | 149-150 |
| 8 | 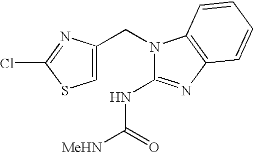 | 192-193 |

### Biological Test On Aphid

### EXAMPLE 9

The compound obtained from Example 1 was mixed with a solvent to form a 9.6% w/w pesticide reagent. An 8 cm×8 cm piece of clean cabbage leaf was placed on a lump of wet cotton in a Petri dish, and then 20 aphids were put on the cabbage leaf in the Petri dish. The thus formed pesticide reagent was diluted 1000 times with water to form a dilution that was then sprayed onto the cabbage leaf in the Petri dish with a predetermined amount. The number of dead aphids was examined 24 hours later, and the kill ratio was calculated. The test was carried out through four replicates.

### EXAMPLES 10 to 16

Example 9 was repeated, except that the compound employed in Example 9 was replaced by the compound obtained from a respective one of the Examples 2 to 8.

### Comparative Example 1

Example 9 was repeated, except that the compound employed in Example 9 was replaced by imidachloprid, a heterocyclic compound of the formula disclosed in U.S. Pat. No. 4,742,060 that can be useful as pesticides.

Table 2 lists the effectiveness of the pesticide reagents prepared in Examples 9 to 16 and the Comparative Example 1.

TABLE 2

| | Number of dead aphids | | | | | |
|---|---|---|---|---|---|---|
| Example | 1st | 2nd | 3rd | 4th | Average | Kill ratio |
| Blank | 1 | 0 | 1 | 0 | 0.5 | — |
| 9 | 19 | 19 | 20 | 20 | 19.25 | 96.15 |
| 10 | 18 | 17 | 18 | 17 | 17.5 | 87.18 |
| 11 | 16 | 19 | 18 | 18 | 17.75 | 88.46 |
| 12 | 20 | 19 | 19 | 19 | 19.25 | 96.15 |
| 13 | 18 | 17 | 16 | 17 | 17 | 84.62 |
| 14 | 17 | 16 | 18 | 16 | 16.75 | 83.33 |
| 15 | 17 | 17 | 16 | 16 | 16.5 | 82.05 |
| 16 | 19 | 16 | 16 | 16 | 16.75 | 83.33 |
| Comparative | 17 | 17 | 18 | 17 | 17.25 | 85.90 |
| Example 1 | | | | | | |

The above biological test results show that the heterocyclic compounds of formula (I) of the present invention exhibit excellent efficiency as pesticides.

While the invention has been described by way of examples, it is to be understood that the invention is not limited thereto. On the contrary, it is apparent that all kinds of modifications and variations can be made without departing from the spirit and the scope of the appended claims.

What is claimed is:

1. A heterocyclic compound having the formula: 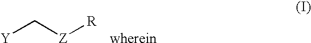
Y represents a 5- or 6-membered nitrogen-containing heterocyclic ring;
Z represents a nitrogen atom, an oxygen atom, a sulfur atom or NR¹;
R represents a hydrogen atom, a halogen atom, a hydroxy group, an alkoxy group, a substituted or unsubstituted amino group, a cyano group, a nitro group, a nitroso group, an alkyl group, an alkenyl group, an alkynyl group, a substituted or unsubstituted phenyl or naphthyl group, an alkylcarbonyl group, an alkenylcarbonyl group, an alkynylcarbonyl group, a substituted or unsubstituted phenylcarbonyl group, an alkoxycarbonyl group, an alkylthiocarbonyl group, a substituted or unsubstituted phenoxycarbonyl group, a substituted or unsubstituted phenylthiocarbonyl group, a monoalkyl- or dialkyl-aminocarbonyl group, a substituted or unsubstituted phenylaminocarbonyl group, a substituted or unsubstituted benzoylaminocarbonyl group, an alkylsulfonyl group, a substituted or unsubstituted phenylsulfonyl group, a substituted or unsubstituted phenylsulfonylaminocarbonyl group, an organophosphono group, an organothionophosphono group, or 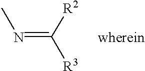
R² and R³ independently represent an alkyl group, an alkenyl group, an alkynyl group, a substituted or unsubstituted phenyl or naphthyl group, an alkoxy group, an alkenyloxy group, an alkynyloxy group, a substituted or unsubstituted phenyloxy or naphthyloxy group, an alkylthio group, an alkenylthio group, an alkynylthio group, a substituted or unsubstituted phenylthio or naphthylthio group, a substituted or unsubstituted amino group;
R¹ represents a hydrogen atom, a halogen atom, a hydroxy group, an alkoxy group, a substituted or unsubstituted amino group, a cyano group, a nitro group, a nitroso group, an alkyl group, an alkenyl group, an alkynyl group, a substituted or unsubstituted phenyl or naphthyl group, an alkylcarbonyl group, an alkenylcarbonyl group, an alkynylcarboyl group, a substituted or unsubstituted phenylcarbonyl group, an alkoxycarbonyl group, an alkylthiocarbonyl group, a substituted or unsubstituted phenoxycarbonyl group, a substituted or unsubstituted phenylthiocarbonyl group, a monoalkyl- or dialkyl-aminocarbonyl group, a substituted or unsubstituted phenylaminocarbonyl group, a substituted or unsubstituted benzoylaminocarbonyl group, an alkylsulfonyl group, a substituted or unsubstituted phenylsulfonyl group, a substituted or unsubstituted phenylsufonylaminocarbonyl group, an organophosphono group, a n organothionophosphono group, 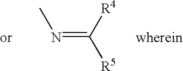
R⁴ and R⁵ independently represent an alkyl group, an alkenyl group, an alkynyl group, a substituted or unsubstituted phenyl or naphthyl group, an alkoxy group, an alkenyloxy group, an alkynyloxy group, a substituted or unsubstituted phenyloxy or naphthyloxy group, an alkylthio group, an alkenylthio group, an alkynylthio group, a substituted or unsubstituted phenylthio or naphthylthio group, a substituted or unsubstituted amino group.

2. 6-Chloro-pyridin-3-ylmethyl-cyanamide according to claim 1 of the formula

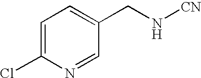

3. N-(6-chloro-pyridin-3-ylmethoxy)-thioacetimidic acid methyl ester according to claim 1 of the formula

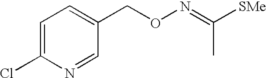

4. 2-Chloro-5 -(2-isopropoxy-phenoxymethyl)-pyridine according to claim 1 of the formula

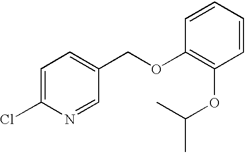

5. 2-chloro-5-(3,5-dimethyl-4-methylsulfanyl-phenoxymethyl)-pyridine according to claim 1 of the formula

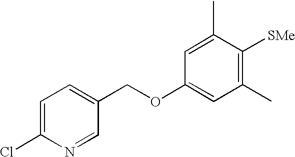

6. 2-Chloro-5-(naphthalen-1-yloxymethyl)-pyridine according to claim 1 of the formula

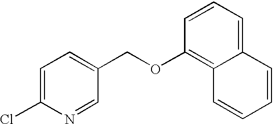

7. 2-chloro-5-(2,2-dimethyl-2,3-dihydro-benzofuran-7-yloxymethyl)-pyridine according to claim 1 of the formula

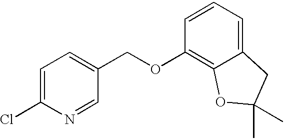

8. 1-[1-(6-Chloro-pyridin-3 -ylmethyl)- 1H-benzoimidazol-2-yl]-3 -methyl-urea according to claim 1 of the formula

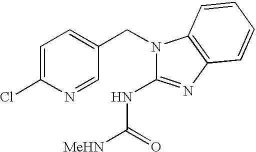

9. 1-[1-(2-Chloro-thiazol-4-ylmethyl)-1H-benzoimidazol-2-yl]-3 -methyl-urea according to claim 1 of the formula

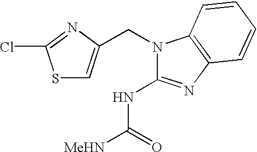

10. A pesticide reagent comprising an effective amount of the hetero cyclic compound of claim 1.

11. A pesticide reagent of claim 10 that further comprises a solvent mixed with an effective amount of the heterocyclic compound of claim 1.
